(19) United States
(12) Patent Application Publication
(10) Pub. No.: US 2008/0234551 A1
(43) Pub. Date: Sep. 25, 2008

(54) Gasless Laparoscopic Retractor Structure

(76) Inventors: Ming-Tsan Lin, Taipei City (TW); Ching-Yao Yang, Taipei City (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 11/690,661

(22) Filed: Mar. 23, 2007

(51) Int. Cl. A61B 1/32

(52) U.S. Cl. 600/235

(57) ABSTRACT

Abdominal annular pack structure for gasless laparotory operation comprises an annular ring, a plurality of hooks and adjusting devices, a fixation ring and a stationary pole with an adjusting rod. The stationary pole is erected on one side of the operation table to settle the adjusting rod thereon in position. The adjusting rod is provided with a clamp at its one end for clutching the fixation ring in position. The fixation ring is disposed to encircle the annular ring surrounding the wound open on the patient belly part. Each adjusting device is connected respectively to its corresponding hook with a pulling string so as to apply a variable tensile force to the annular ring thereby able to adjust the size and shape of the wound. With this structure, the surgical operation may be carried out securely and perfectly without filling the gas (CO₂) to the abdominal cavity, and absolutely getting rid of the fear of any attack of complication to the patient.

### BACKGROUND OF THE INVENTION

1. Field of the Invention

The present invention relates to abdominal annular pack structure for gasless laparotomy operation, and more particularly to an annular pack structure wherein a plurality of evenly arranged retraction hooks on an annular ring are used to part the operation wound on the patient abdomen with an adjusting device to keep a proper size and shape of the wound so as to facilitate performing gasless laparotomy operation.

2. Description of the Prior Art

The traditional laparotomy operation needs to open a rather lengthy opening on the patient's abdomen for surgical procedure which can cause prolonged pain, healing, and post-operative maintenance of the wound. By this reason, an improved method of abdominal operation has been developed in which the abdominal cavity is inflated by gas so as to facilitate examining of the abdominal region with a laparotomy camera by way of laparoscopy before operation.

However, such a simple procedure of filling the gas into the abdominal cavity is by no means free from a latent danger. First of all, it brings about an increased abdominal pressure which not only causes the patient to feel unpleasant, but also affects the functions of the patient's heart and lungs, blood pH value, and basal metabolic rate. In the worst case, severe complication can be fatal to the elderly and children.

Although minimally-invasive laparotomy surgical techniques that inflates the abdominal region offers a smaller incision, it is also more difficult to perform, and carries more potential complications. In addition, in such surgery where the abdomen is filled with gas, it is also harder for the surgeon to get a feel of relative positioning of surgical instruments in the abdomen. For these defects inherent in the prior techniques of the laparotomy operation, an improvement is seriously required.

The inventor of the present invention has devoted years of efforts into this matter in studying and improving the aforementioned defects to come up with a novel abdominal annular pack structure for gasless laparotomy operation as provided in this invention to eliminate the defects mentioned above.

All referenced patents, applications and literatures are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply. The invention may seek to satisfy one or more of the above-mentioned desire. Although the present invention may obviate one or more of the above-mentioned desires, it should be understood that some aspects of the invention might not necessarily obviate them.

### SUMMARY OF THE INVENTION

It is the main object of the preset invention to provide abdominal annular pack structure for gasless laparotomy operation to replace traditional gas CO₂-filling abdominal operation wherein a mechanical annular pack structure is employed to adjust the size and shape of the operation wound (or opening) on the patient's abdomen so as to facilitate the surgeon to observe and inspect the abdominal cavity before operation, and to facilitate surgical operation of the abdomen.

It is another object of the present invention that the conventional instrumentation of the abdominal surgery can be used in collaboration with the new device of the present invention to perform operation combining advantages offered by both. The present invention provides relative easy, secure, convenient, and relatively small wound which heals faster, and results in minimal post-operation emotional trauma usually found in patients with rather large scars from similar surgeries.

To achieve the aforesaid objects, the present invention provides an abdominal annular pack structure including an annular ring, a plurality of hooks, a plurality of adjusting devices, a fixation ring, and a stationary pole with an adjusting rod. The stationary pole is erected on one side of the operation table to settle the adjusting rod thereon in position. The adjusting rod is provided with a clamp at one end thereof for clutching the fixation ring in position. The fixation ring is disposed to encircle the annular ring surrounding the wound open on the patient's abdominal region. Each adjusting device is connected respectively to the corresponding retraction hook with a pulling string so as to apply a variable tensile force to the annular ring thereby adjusting the size and shape of the wound. With this structure, the operation may be carried out securely and perfectly without filling gas into the abdominal cavity. Instead, the surgical opening on the patient's body is directly and adjustably held open using mechanical parts, allowing the surgeon to control the size of the opening for ease of operation, and decreases the likelihood of post-operative complication.

### BRIEF DESCRIPTION OF THE DRAWINGS

The drawings disclose an illustrative embodiment of the present invention which severs to exemplify the various advantages and objects hereof, and are as follows:

FIG. 1 is an illustrative view in an embodiment of the present invention;

FIG. 2 is a schematic structural view of the annular ring;

FIG. 3A, B, C, D show schematic structural views of the hook;

FIG. 4 is a schematic structural view of the adjustor;

FIG. 5 is a schematic structural view of the fixation ring;

FIG. 6 is an illustrative view showing the structural layout and positional relation between the fixed ole and the adjusting rod; and

FIG. 7 is a complete assembly view of the present invention.

### DETAILED DESCRIPTION OF THE PREFERRED EMBODIMENT

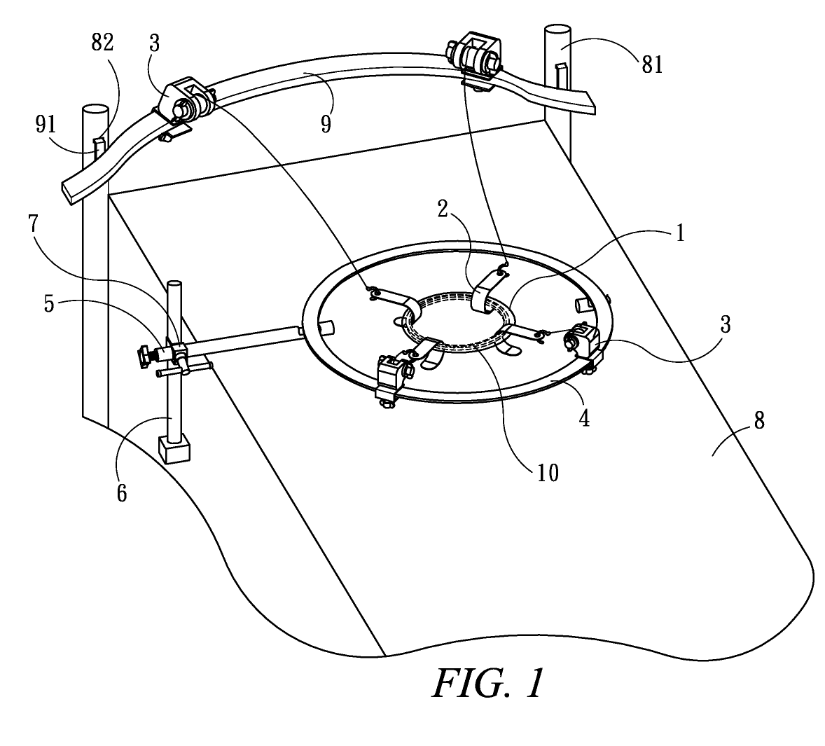

Referring to FIG. 1, the abdominal annular pack structure of the present invention comprises an annular ring 1, a plurality of retraction hooks 2, a plurality of adjusting device 3, a fixation ring 4, and a stationary pole 6 with an adjusting rod 5. The annular ring 1 is disposed to surround the wound opening on the patient's belly for surgery. The hooks 2 are disposed evenly along the inner edge of the annular ring 1, and each of the hooks 2 is connected respectively to the corresponding adjusting device 3 with a pulling string 31 so as to vary the opened diameter of the annular ring 1. Each of the adjusting device 3 is installed on the fixation ring 4 which is fixedly clamped in position by the adjusting rod 5. The adjusting rod 5 is attached to the stationary pole 6. With such arrangement, a secure and easy laporotomy surgical spacing is formed for the surgeon to perform his work.

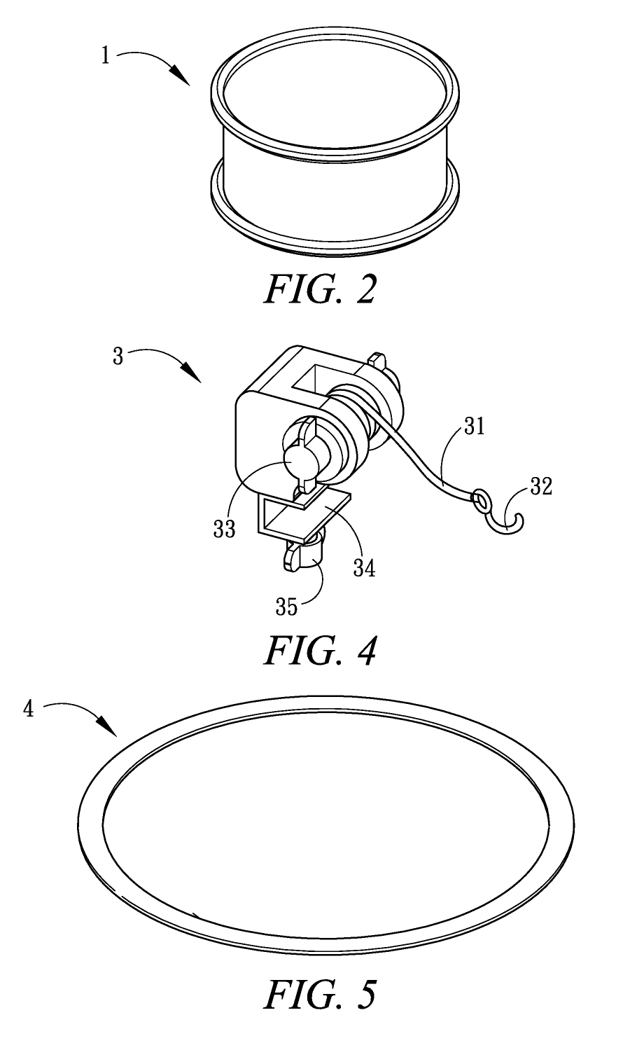

Referring to FIG. 2, the annular ring 1 is an elastic structure formed of a resilient substance convenient to adjust its size and shape to meet the requirement for surgical operation. Besides, the annular ring 1 may protect the wound from direct contact with the hook 2 so as to prevent possible infection, electro-burning, or metastasis of cancer cells via open wound.

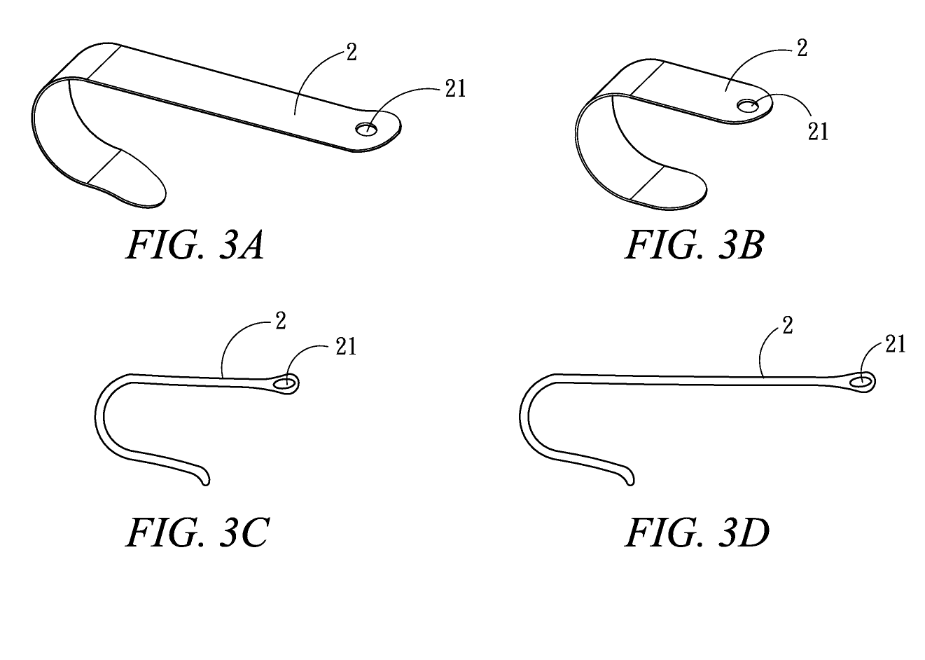

Referring to FIGS. 3A to 3D, the hook 2 may be configured into various suitable size and shape in accordance with the patient's belly shape, thickness of the abdominal wall, relative location of the opening on the body, size of opening, to facilitate the intended operation. An eye hole 21 is provided at a terminal for holding the pulling string 31 which interconnects the hook 2 and its adjusting device 3.

Referring to FIG. 4, the string 31 is connected to the hook 2 with another hook 32 engaged with the eye hole 21. The adjusting device 3 is provided with an adjusting screw 33 to adjust the stretched length of the string 31 which in turn varies the size of the annular ring 1. A rail rest 34 is provided at the lower end of the adjusting device 3 to enclose the fixation ring 4 and enable the adjusting device 3 to move along the fixation ring 4. By way of tightening a fixing screw 35 provided at the bottom of the rail rest 31, the adjusting device 3 can be set at a desired position on the fixation ring 4.

Referring to FIG. 5, the fixation ring 4 used to dispose and settle the adjusting device 3 is made in a flat plate configuration so as to allow easy clamping by the rail rest 34 of the adjusting device 3.

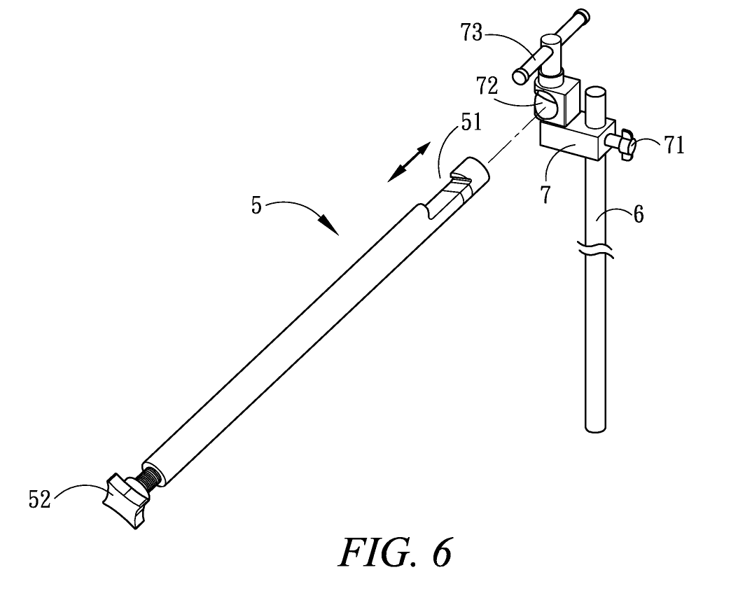

Referring to FIG. 6, a clamp 51 is provided at one end of the adjusting rod 5 to clutch the fixation ring 4. The opening of the clamp 51 is controlled by an adjusting screw 52 provided at the other end of the adjusting rod 5 so as to tightly clutch the fixation ring 4. The stationary pole 6 is provided with a fixing device 7 which can be settled at a desired position on the stationary pole 6 with a fixing screw 71. A pinch 72 provided for the fixing device 7 is used to set the fixing device 7 on the adjusting rod 5 so as to adjust the position of the adjusting rod 5 with respect to the stationary pole 6. After finishing adjustment, the adjusting rod 5 is fixed in position with an fixing screw 73. By doing so, the adjusting rod 5 is operable in a region defined by the stationary pole 6, and the elevation of the adjusting rod 5 is regulated by the fixing device 7.

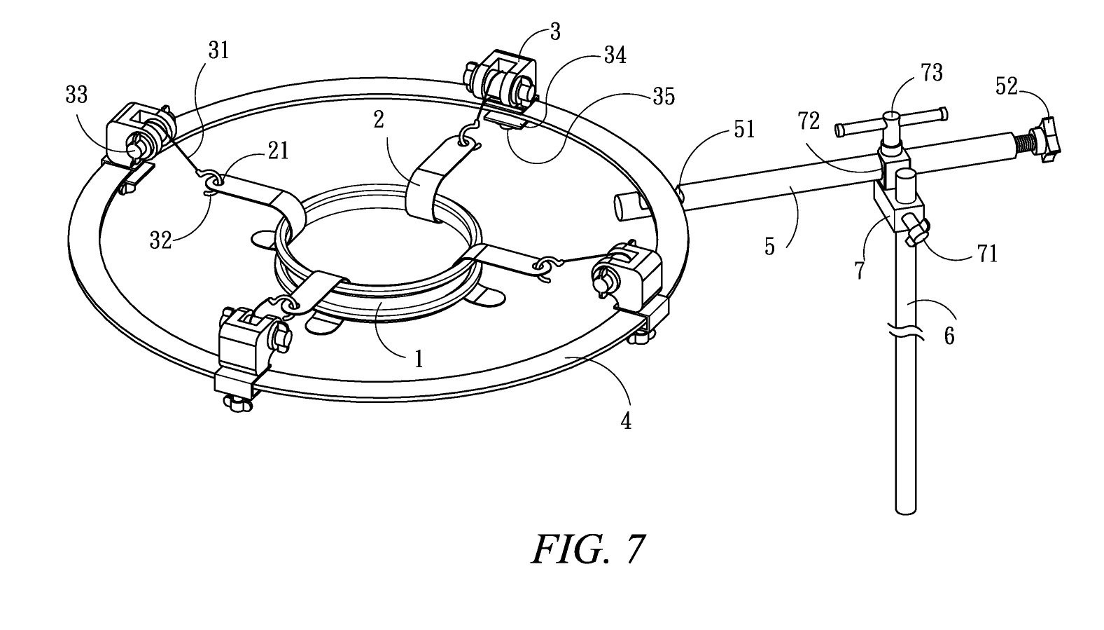

Referring to FIG. 7 together with FIG. 1, at the beginning of the surgery, the stationary pole 6 is erected near the operation table, or alternatively, it can be erected at another desirable location in the operation room so long as the location offers stable and secure installation. Once the stationary pole 6 is securely installed, the adjusting rod 5 is then secured onto the stationary pole 6, and then the fixation ring 4 is fixed in a desired position by the adjusting rod 5. After that, the annular ring with the hooks 2 are set around the region to be operated on, then the adjusting devices 3 are disposed appropriately along the fixation ring 4, and the opening diameter of the annular ring 1 is adjusted by controlling the tensile force of the strings 31 hanging between the hooks 2 and the adjusting devices 3. For further protection of the patient, an optional inner coaxial protective ring 10 can be provided to avoid the direct contact of the hooks 2 with the wall of the wound, or possible deformation of the annular ring 1 during application of pulling force.

In other preferred embodiments, for convenient setting of the adjusting devices 3, a Kentledge bridge 9 may be established. A hanger 91 is provided at each of the two sides of the bridge 9, and a corresponding guide slot 82 is formed at each of the two feet 81 of the operation table 8 so as to hook the hanger 91 respectively onto its corresponding guide slot 82. By doing so, the adjusting devices 3 may be either disposed along the fixation ring 4, or on the bridge 9. With such dual positioning structure of the table feet 81 and the stationary pole 6, the stability of the present invention can be further ensured.

Many changes and modifications in the above described embodiment of the invention can, of course, be carried out without departing form the scope thereof. Accordingly, to promote the progress in science and the useful arts, the invention is disclosed and is intended to be limited only by the scope of the appended claims.

What is claimed is:

1. Abdominal annular pack structure for gasless laparotomy operation comprising:
an annular ring surrounding the wound opened on the patient belly for surgery;
a plurality of retraction hooks disposed evenly along the inner edge of said annular ring;
a plurality of adjusting devices, each of them connected to a corresponding hook respectively so as to adjust the size of said wound by pulling said hook;
a fix ring encircling said annular ring for installing said adjusting devices;
an adjusting rod having a clamp at its one end to clutch said fixation ring; and
a stationary pole being attached by said adjusting rod and serving as a supporter of the entire structure.

2. The structure of claim 1, wherein said annular ring is an elastic structure capable of adjusting its size and shape to meet the requirement for surgical operation.

3. The structure of claim 1, wherein said hook is configurated into various sizes and shapes in accordance with the patient's belly shape to facilitate surgical operation.

4. The structure of claim 1, wherein said adjusting device is provided with a pulling string, while said hook has an eye hole formed at its terminal, by engaging another hook provided at the end of said pulling string with said eye hole, the stretched length of said string can be controlled by an adjusting screw provided at the side of said adjusting device so as to vary the size of said annular ring.

5. The structure of claim 1, wherein said adjusting device is provided with a rail rest at the lower end thereof to enclose said fixation ring and enable said adjusting device to move therealong, by tightening a fixing screw provided at the bottom of said rail rest, said adjusting device is set at a desired position on said fixation ring.

6. The structure of claim 1, wherein a Kentledge bridge is established for said structure for settling said adjusting devices, a hanger is provided at each of the two sides of said bridge, and a corresponding guide slot 82 is formed at each of two feet of the operation table so as to hook said hanger respectively onto its corresponding slot.

7. The structure of claim 1, wherein said annular ring is provided with an inner coaxial protective ring.

8. The structure of claim 1, wherein said stationary pole is erected by the operation table, or on another desirable location in the operation room.

9. The structure of claim 1, wherein said stationary pole is provided with a fixing device which can be settled at a desired position on said stationary pole with a fixing screw, a pinch is provided for said fixing service to set it on said adjusting rod so as to adjust the position of said adjusting rod relative to said stationary pole, after finishing adjustment, said adjusting rod is fixed at position with an fixing screw.

10. The structure of claim 1, wherein the opening of said clamp provided for said adjusting rod is controllable with an adjusting screw so as to tightly clutch said fixation ring.
